(19) United States
(12) Patent Application Publication
(10) Pub. No.: US 2017/0251965 A1
(43) Pub. Date: Sep. 7, 2017

(54) BLOOD SAMPLING DEVICE

(71) Applicant: MEDIFUN CORPORATION, Taichung City (TW)

(72) Inventors: William Chen, Taichung City (TW); Hao-Cheng Fan, Taichung City (TW)

(21) Appl. No.: 15/062,190

(22) Filed: Mar. 6, 2016

(51) Int. Cl. A61B 5/15; A61B 5/151

(52) U.S. Cl. CPC A61B 5/150183; A61B 5/150824; A61B 5/15132

(57) ABSTRACT

A blood sampling device contains: a body, a fitting sleeve, and an adjusting sleeve. The body includes a lancet, a driving edge for driving the lancet, a control switch for controlling the driving edge, and a connecting section. The fitting sleeve includes a first fitting section, a second fitting section, and a connecting groove, wherein the second fitting section has a coupling portion for connecting with the connecting section. The fitting sleeve further includes a trench, an outlet, and a fixing rib. The adjusting sleeve includes a coupling section, a controlling section opposite to the coupling section, a tilted guiding slot for retaining with the fixing rib, and an exit corresponding to the outlet, The second fitting section of the fitting sleeve has a transparent window for viewing the coupling section of the adjusting sleeve, and the coupling section has a length identification portion injection molded thereon.

### FIELD OF THE INVENTION

The present invention relates to a blood sampling device which is capable of adjusting an extending length of a lancet as sampling patient's blood.

### BACKGROUND OF THE INVENTION

A conventional blood sampling device is employed to sample patient's bloods after inserting its lancet into patient's skin so as to test blood sugar from blood sample, and an extending length of a lancet into the patient's ski therefore determines sampled volume of the patient's blood. To distinguish the extending length of the lancet, a length identification portion is printed on a scale section of an adjusting sleeve of the conventional blood sampling device, but such a printing manner increases production process.

The present invention has arisen to mitigate and/or obviate the afore-described disadvantages.

### SUMMARY OF THE INVENTION

The primary objective of the present invention is to provide a blood sampling device which is capable of adjusting an extending length of a lancet as sampling patient's blood.

Another objective of the present invention is to provide a blood sampling device which contains a length identification portion 36 injection molded on the coupling section so as to simplify production process.

To obtain the above objectives, a blood sampling device provided by the present invention contains: a body, a fitting sleeve, and an adjusting sleeve.

The body includes a lancet accommodated therein, a driving edge for driving the lancet to move outwardly, a control switch for controlling the driving edge, and a connecting section defined between the driving edge and the control switch and fitting with the fitting sleeve.

The fitting sleeve includes a first fitting section and a second fitting section, wherein a part of the first fitting section is fitted with the second fitting section, and the fitting sleeve includes a connecting groove formed between the first fitting section and the second fitting section and configured to accommodate the adjusting sleeve, the second fitting section has a coupling portion arranged on one end thereof so as to connect with the connecting section, the fitting sleeve further includes a trench defined on a central position of the first fitting section thereof so that the lancet moves in the trench, the fitting sleeve further includes an outlet defined on one end of the first fitting section thereof so as to extend the lancet out of the outlet, and the fitting sleeve further includes a fixing rib formed on an inner wall of the first fitting sleeve.

The adjusting sleeve includes a coupling section inserting into the connecting groove, a controlling section opposite to the coupling section, a tilted guiding slot defined on an outer wall of the coupling section so as to retain with the fixing rib, and an exit formed on outer end of the controlling section and corresponding to the outlet.

The second fitting section of the fitting sleeve has a transparent window fixed thereon so as to view the coupling section of the adjusting sleeve, and the coupling section has a length identification portion injection molded thereon.

Preferably, the fitting sleeve further includes at least one elastic retainer arranged on an outer wall of the first fitting sleeve, and the adjusting sleeve further includes a plurality of limiting recesses for selectively retaining with the at least one elastic retainer.

Preferably, the body further includes plural locking notches arranged on the connecting section, and the fitting sleeve further includes a plurality of protrusions arranged on an inner wall of the coupling portion so as to lock with the plural locking notches, respectively.

Preferably, a diameter of the exit is less than the outlet, and the exit has a casing extending inwardly so as to shield the outlet.

### BRIEF DESCRIPTION OF THE DRAWINGS

FIG. 1 is a cross-sectional perspective view showing the exploded components of a blood sampling device according to a preferred embodiment of the present invention.

FIG. 2 is a perspective view showing the exploded components of a part of the blood sampling device according to the preferred embodiment of the present invention.

FIG. 3 is a perspective view showing the assembly of the blood sampling device according to the preferred embodiment of the present invention.

FIG. 4 is a side plane view showing the operation of the blood sampling device according to the preferred embodiment of the present invention.

FIG. 5 is a cross sectional view taken along the line A-A of FIG. 4.

FIG. 6 is another side plane view showing the operation of the blood sampling device according to the preferred embodiment of the present invention.

FIG. 7 is a cross sectional view taken along the line A-A of FIG. 6.

FIG. 8 is also another side plane view showing the operation of the blood sampling device according to the preferred embodiment of the present invention.

FIG. 9 is a cross sectional view taken along the line A-A of FIG. 8.

### DETAILED DESCRIPTION OF THE PREFERRED EMBODIMENTS

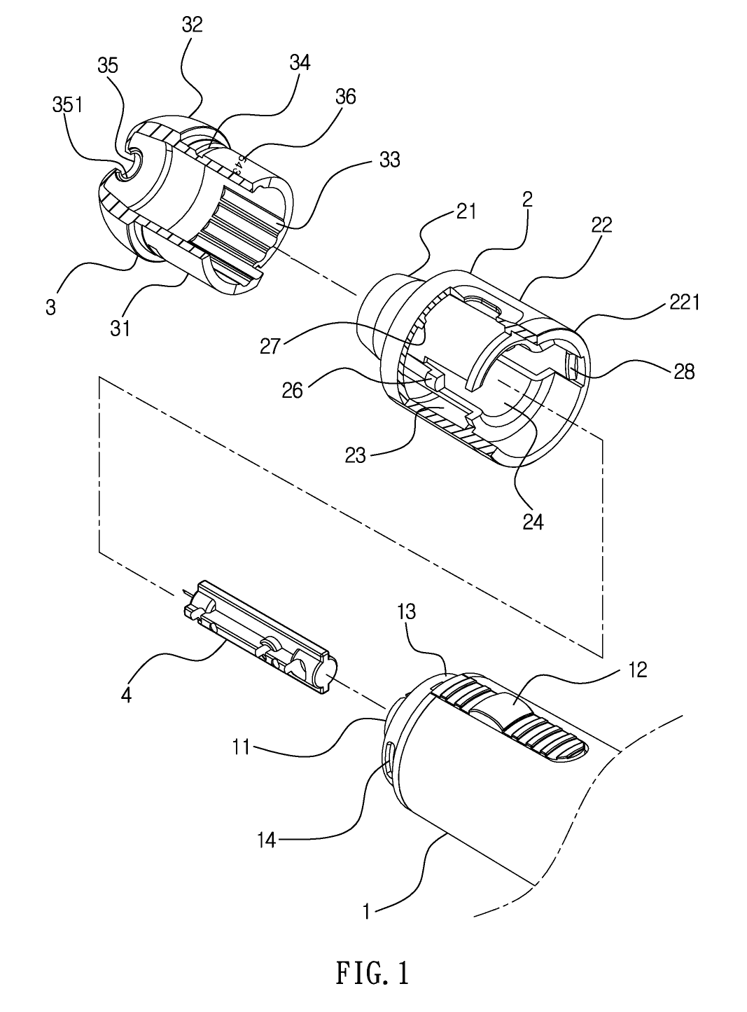

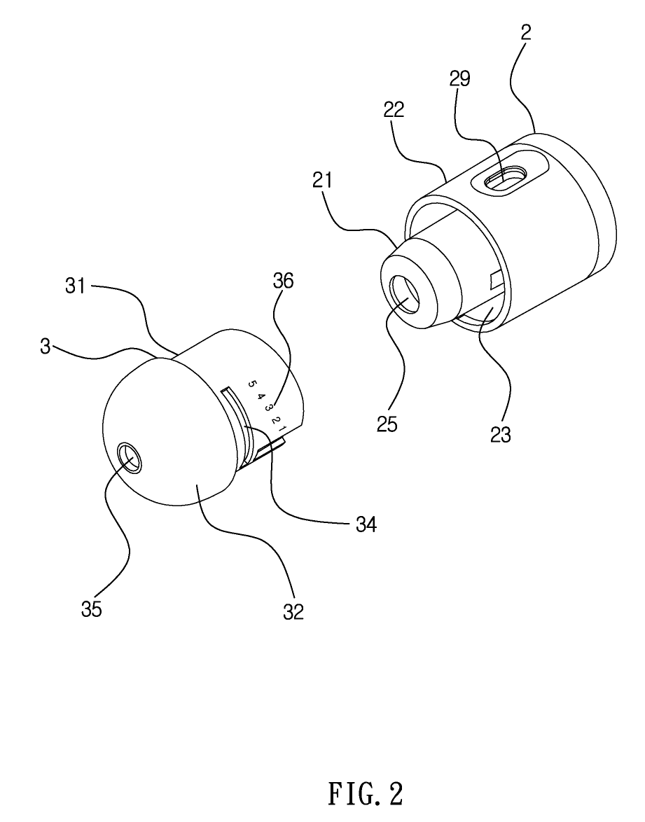

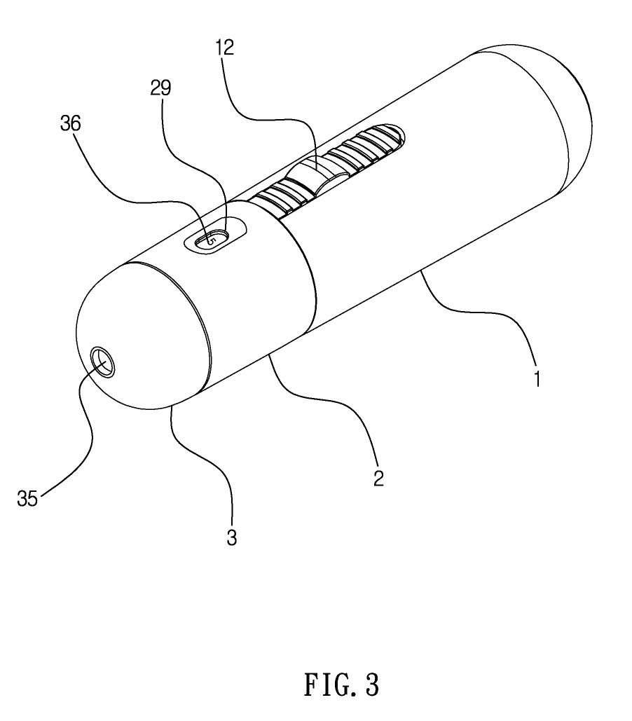

With reference to FIGS. 1 to 3, a blood sampling device according to a preferred embodiment of the present invention is capable of adjusting an extending length of a lancet as sampling patient's blood and comprises: a body 1, a fitting sleeve 2, and an adjusting sleeve 3.

The body 1 includes a lancet 4 accommodated therein, a driving edge 11 for driving the lancet 4 to move outwardly, a control switch 12 for controlling the driving edge 11, and a connecting section 13 defined between the driving edge 11 and the control switch 12 and fitting with the fitting sleeve 2. The lancet 4 is configured to sample the patient's blood and is a well-known art, so its further remarks are omitted.

The fitting sleeve 2 includes a first fitting section 21 and a second fitting section 22, wherein a part of the first fitting section 21 is fitted with the second fitting section 22, and the fitting sleeve 2 further includes a connecting groove 23 formed between the first fitting section 21 and the second fitting section 22 and configured to accommodate the adjusting sleeve 3, the second fitting section 22 has a coupling portion 221 arranged on one end thereof so as to connect with the connecting section 13. The fitting sleeve 2 further includes a trench 24 defined on a central position of the first fitting section 21 thereof so that the lancet 4 moves in the trench 24. The fitting sleeve 2 further includes an outlet 25 defined on one end of the first fitting section 21 thereof so as to extend the lancet 4 out of the outlet 25, such that the lancet 4 is driven by the driving edge 11 to move in the trench 24 and its head end extends out of the outlet 25. The fitting sleeve 2 further includes at least one elastic retainer 26 arranged on an outer wall of the first fitting sleeve 21 and includes a fixing rib 27 formed on an inner wall of the first fitting sleeve 21.

The adjusting sleeve 3 includes a coupling section 31 inserting into the connecting groove 23, a controlling section 32 opposite to the coupling section 31, a plurality of limiting recesses 33 for selectively retaining with the at least one elastic retainer 26, a tilted guiding slot 34 defined on an outer wall of the coupling section 31 so as to retain with the fixing rib 27, and an exit 35 formed on outer end of the controlling section 32 and corresponding to the outlet 25.

The body 1 further includes plural locking notches 14 arranged on the connecting section 13, and the fitting sleeve 2 further includes a plurality of protrusions 28 arranged on an inner wall of the coupling portion 221 so as to lock with the plural locking notches 14, respectively, such that the fitting sleeve 2 and the adjusting sleeve 3 are connected with and removed from the body 1 quickly.

The second fitting section 22 of the fitting sleeve 2 has a transparent window 29 fixed thereon so as to view the coupling section 31 of the adjusting sleeve 3, and the coupling section 31 has a length identification portion 36 injection molded thereon so that the extending length of the lancet 4 identified by the length identification portion 36 is visible through the transparent window 29.

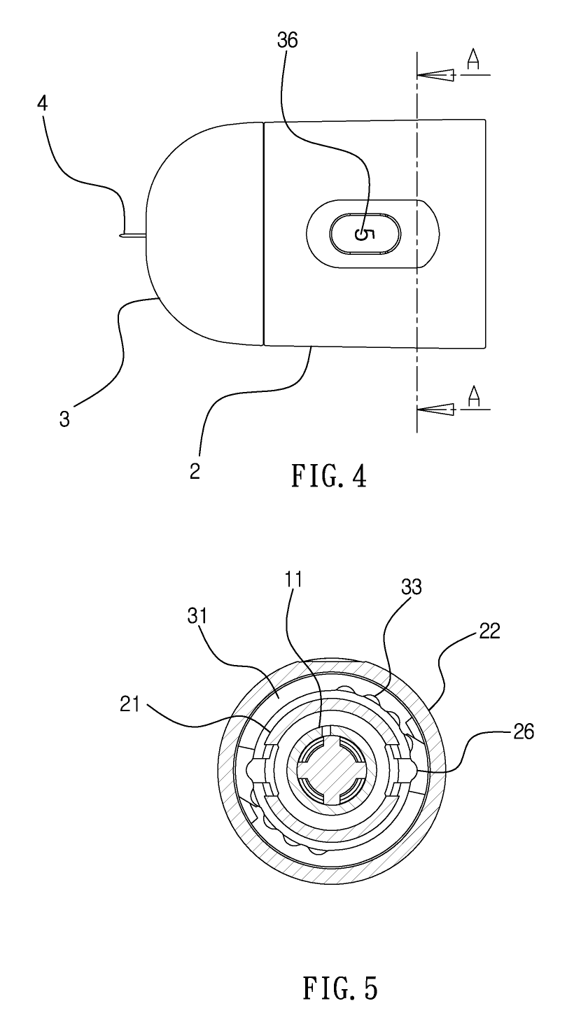

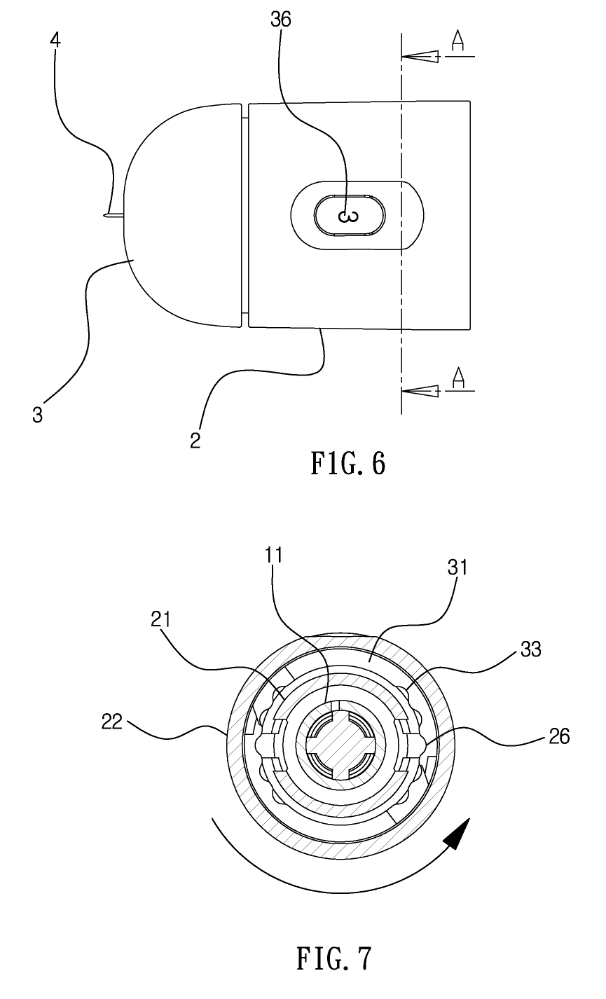

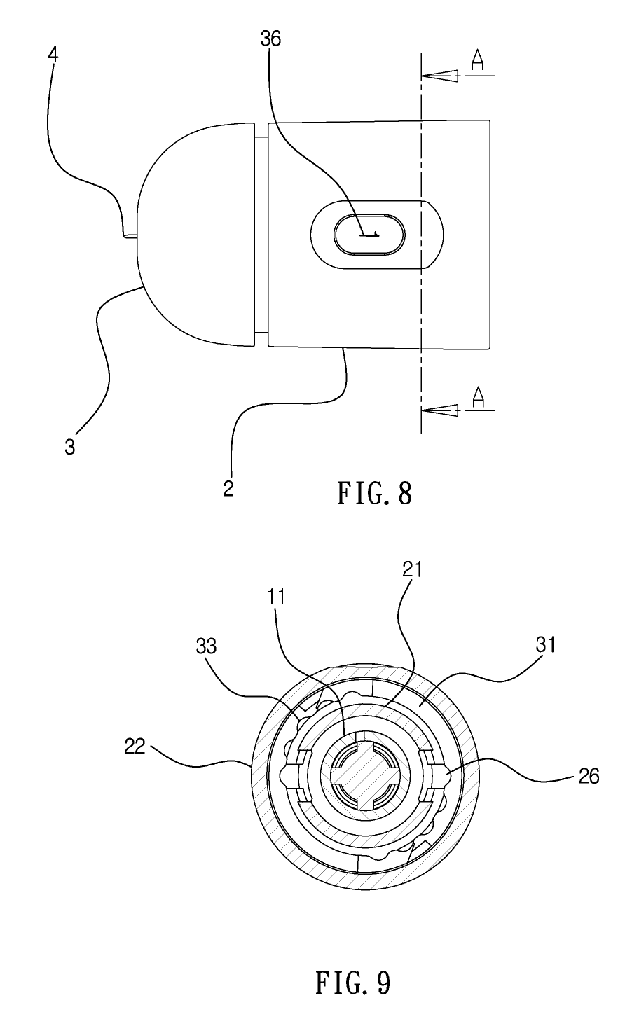

Referring to FIGS. 4 to 9, as adjusting the extending length of the lancet 4, at least one of the coupling sleeve 2 and the adjusting sleeve 3 is rotated. For example, the adjusting sleeve 3 is rotated relative to the fitting sleeve 2 so that the at least one elastic retainer 26 moves among the plurality of limiting recesses 33 so as to fix the adjusting sleeve 3 at a position of the fitting sleeve 2, and the tilted guiding slot 34 is moved relative to the fixing rib 27 so that a distance between the adjusting sleeve 3 and the fitting sleeve 2 is adjusted, and a distance between the exit 35 and the outlet 25 is adjustable as well.

It is to be noted that a diameter of the exit 35 is less than the outlet 25, and the exit 35 has a casing 351 extending inwardly so as to shield the outlet 25, thus stopping the patient's blood flowing into the fitting sleeve 2.

Thereby, the at least one of the adjusting sleeve 3 and the fitting sleeve 2 of the blood sampling structure of the present invention is rotated so as to adjust the distance between the exit 35 and the outlet 25, wherein when the distance between the exit 35 and the outlet 25 is prolonged, the extending length of the lancet 4 out of the exit 35 is shorten, and when the distance between the exit 35 and the outlet 25 is shorten, the extending length of the lancet 4 out of the exit 35 is prolonged, thus sampling the patient's blood based on using requirement. Preferably, the length identification portion 36 is injection molded on the coupling section 31 so as to simplify production process.

While the preferred embodiments of the invention have been set forth for the purpose of disclosure, modifications of the disclosed embodiments of the invention and other embodiments thereof may occur to those skilled in the art. Accordingly, the appended claims are intended to cover all embodiments which do not depart from the spirit and scope of the invention.

What is claimed is:

1. A blood sampling device comprising: a body, a fitting sleeve, and an adjusting sleeve; wherein
the body includes a lancet accommodated therein, a driving edge for driving the lancet to move outwardly, a control switch for controlling the driving edge, and a connecting section defined between the driving edge and the control switch and fitting with the fitting sleeve;
the fitting sleeve includes a first fitting section and a second fitting section, wherein a part of the first fitting section is fitted with the second fitting section, and the fitting sleeve includes a connecting groove formed between the first fitting section and the second fitting section and configured to accommodate the adjusting sleeve, the second fitting section has a coupling portion arranged on one end thereof so as to connect with the connecting section, the fitting sleeve further includes a trench defined on a central position of the first fitting section thereof so that the lancet moves in the trench, the fitting sleeve further includes an outlet defined on one end of the first fitting section thereof so as to extend the lancet out of the outlet, and the fitting sleeve further includes a fixing rib formed on an inner wall of the first fitting sleeve;
the adjusting sleeve includes a coupling section inserting into the connecting groove, a controlling section opposite to the coupling section, a tilted guiding slot defined on an outer wall of the coupling section so as to retain with the fixing rib, and an exit formed on outer end of the controlling section and corresponding to the outlet;
the second fitting section of the fitting sleeve has a transparent window fixed thereon so as to view the coupling section of the adjusting sleeve, and the coupling section has a length identification portion injection molded thereon.

2. The blood sampling device as claimed in claim 1, wherein the fitting sleeve further includes at least one elastic retainer arranged on an outer wall of the first fitting sleeve, and the adjusting sleeve further includes a plurality of limiting recesses for selectively retaining with the at least one elastic retainer.

3. The blood sampling device as claimed in claim 1, wherein the body further includes plural locking notches arranged on the connecting section, and the fitting sleeve further includes a plurality of protrusions arranged on an inner wall of the coupling portion so as to lock with the plural locking notches, respectively.

4. The blood sampling device as claimed in claim 1, wherein a diameter of the exit is less than the outlet, and the exit has a casing extending inwardly so as to shield the outlet.
